(19) United States
(12) Patent Application Publication
(10) Pub. No.: US 2026/0094706 A1
(43) Pub. Date: Apr. 2, 2026

(54) BUILDING SYSTEMS FOR EFFICIENT TEMPERATURE, PRESSURE, AND HUMIDITY COMPLIANCE IN HEALTHCARE FACILITIES

(71) Applicant: Tyco Fire & Security GmbH, Neuhausen am Rheinfall (CH)

(72) Inventors: Kyle Shamrock, Glenview, IL (US); Louis Nunzio Loizides, Safety Harbor, FL (US); Ashvin Suthar, Pune (IN); Domenic Busa, Milwaukee, WI (US); Brian D. Troup, Milwaukee, WI (US)

(21) Appl. No.: 19/341,939

(22) Filed: Sep. 26, 2025

(30) Foreign Application Priority Data
Sep. 27, 2024 (IN) 202441073128

(51) Int. Cl. G16H 40/40; F24F 11/50; F24F 11/66; F24F 110/10; F24F 110/20; F24F 120/10

(52) U.S. Cl. CPC G16H 40/40; F24F 11/50; F24F 11/66; F24F 2110/10; F24F 2110/20; F24F 2120/10

(57) ABSTRACT

A system for climate control of an operating room at a healthcare facility includes building equipment operable to affect at least one of temperature, pressure, airflow, or humidity of the operating room and a control system programmed to generate, based on a converged schedule for the operating room, a setback period during which the building equipment for the operating room can be operated in a setback state and control the building equipment using the converged schedule such that the building equipment operates in the setback state during the setback period.

### CROSS-REFERENCE TO RELATED APPLICATIONS

This patent application claims the benefit of and priority to Indian Provisional Patent Application No. 202441073128 filed Sep. 27, 2024, the entire disclosure of which is incorporated by reference herein.

### BACKGROUND

The present disclosure relates to systems and methods of efficiently controlling temperature, humidity, pressure, and airflow within a room and/or a building. In some cases, temperature, pressure, humidity, and airflow (e.g., air changes, air change rate) for an operating room are monitored and maintained in compliance with regulations or process controls. These environmental conditions for an operating room can be maintained in compliance at all times but will include higher costs as a result.

In some embodiments, particularly for a building that serves as a hospital, The Joint Commission (TJC) may administer the compliance checks. In some such embodiments, if the hospital building or a room within the building (e.g., a patient room, an operating room, etc.) is found to be out of compliance, a finding is identified and reported to the Centers for Medicare and Medicaid Services (CMS), who then perform an independent inspection of the building or room. A finding may impact the hospital's ratings, funding, etc., and correcting compliance issues may be expensive and time consuming. Over time, if a hospital regularly fails CMS inspections and/or multiple rooms or devices of the building are regularly non-compliant, a deemed status of the hospital may be lost. The loss of deemed status can result in the withholding of Medicare and/or Medicaid reimbursement to the hospital. In a hospital setting, response to issues affecting environmental conditions in a timely manner is critical. A system for monitoring environmental conditions and related factors could improve a hospital's ability to pass inspections and maintain a healthy environment for patient care. However, always maintaining the environmental conditions of an operating room at all times, for example including through times when the operating room will not be in use for several consecutive hours, can correspond to significant energy consumption and associated operation costs for that operating room.

### SUMMARY

One implementation of the present disclosure is a system for climate control of an operating room at a healthcare facility. The system includes building equipment operable to affect at least one of temperature, pressure, airflow or humidity of the operating room, an occupancy sensor configured to provide occupancy data indicative of whether the operating room is occupied, and a control system. The control system is programmed to generate, based on a surgery schedule for the operating room, standard facility open hours for the healthcare facility, and the occupancy data, a converged schedule for the operating room, the converged schedule indicating setback period during which the building equipment for the operating room can be operated in a setback state for the at least one of temperature, airflow, or humidity of the operating room. The control system is also programmed to control the building equipment using the converged schedule such that the building equipment operates to achieve the compliance requirements for the at least one of temperature, pressure, airflow, or humidity of the operating room other than during the setback period.

In some embodiments, the control system is programmed to control the building equipment based on the converged schedule by causing the building equipment to achieve a first requirement for airflow of an operating room other than during the setback period and to achieve a second requirement for the airflow of the operating room during the setback period.

In some embodiments, the control system is programmed to generate the converged schedule by excluding, from the setback period first times for which the converged schedule indicates that a surgery is scheduled to be performed in the operating room, second times corresponding to the standard facility open hours, third times at which the occupancy data indicates the operating room is occupied, and fourth times for which the system health (i.e. the operating state of the sensors, the operating state of the OpenBlue Bridge software, and the integration of the surgery schedule) is determined to be unhealthy.

In some embodiments, the control system is further programmed to automatically determine a preconditioning requirement for returning the operating room to a non-setback state following the setback period and shorten the setback period in the converged schedule based on the preconditioning requirement.

In some embodiments, the system also includes an override device. The override device is configured to communicate directly with the building management system (BMS). In response to receiving an indication from the override device, the BMS overrides the converged schedule. The override device may be a thermostat interface located in the operating room.

In some embodiments, the control system is further programmed to generate the converged schedule based on system health data associated with the building equipment. The control system may be programmed to dynamically update the converged schedule based on changes in the occupancy data. The control system is further programmed to generate a report comprising information on compliance with the compliance requirements and energy savings associated with the setback period.

In some embodiments, the control system is programmed to control the building equipment using the converged schedule by reducing an airflow provided by the building equipment during the setback period as compared to at times outside the setback period. The control system is programmed to control the building equipment using the converged schedule by using a lower temperature setpoint during the setback period as compared to outside the setback period when the building equipment is heating the operating room and using a higher temperature setpoint during the setback period as compared to outside the setback period when the building equipment is cooling the operating room.

Another implementation of the present disclosure is a method for a healthcare space. The method includes operating building equipment to affect at least one of temperature, pressure, airflow, or humidity of the healthcare space, generating, based on a treatment schedule for the healthcare space, standard open hours for the healthcare space, and sensed occupancy relating to the healthcare space, a converged schedule for the healthcare space by providing the converged schedule with a setback period during which the building equipment of the healthcare space can be operated in a setback state for the at least one of temperature, airflow, or humidity of the healthcare space. The method also includes operating, by the building equipment, in accordance with the converged schedule such that the healthcare space complies with the compliance requirements for the at least one of temperature, pressure, airflow, or humidity of the healthcare space other than during the setback period.

In some embodiments, the method includes generating the converged schedule by excluding, from the setback period, first times for which the treatment schedule indicates that a treatment is scheduled to be performed in the healthcare space, second times corresponding to the standard open hours, third times at which the sensed occupancy indicates the healthcare space is occupied, and fourth times for which the system health is determined to be unhealthy. In some embodiments, generating the converged schedule includes automatically determining a preconditioning requirement for returning the healthcare space to a non-setback state following the setback period and shortening the setback period in the converged schedule based on the preconditioning requirement.

In some embodiments, generating the converged schedule is based on system health data associated with the building equipment. In some embodiments, the method includes dynamically updating the converged schedule based on changes in the sensed occupancy.

In some embodiments, the method includes automatically generating a report of compliance with the compliance requirements and energy saves associated with the setback period.

Operating, by the building equipment, in accordance with the converged schedule may include turning off at least a portion of the building equipment for the setback period. In some scenarios, operating the building equipment to affect the at least one of temperature, pressure, airflow, or humidity of the healthcare space includes heating the healthcare space and operating, by the building equipment, in accordance with the converged schedule comprises using a lower temperature setpoint during the setback period as compared to outside the setback period. In some scenarios, operating the building equipment to affect the at least one of temperature, pressure, airflow, or humidity of the healthcare space includes cooling the healthcare space, and operating, by the building equipment, in accordance the converged schedule comprises using a higher temperature setpoint during the setback period as compared to outside the setback period.

One or more non-transitory computer-readable media storing program instructions that, when executed by one or more processors, cause the one or more processors to perform operations that include generating a converged schedule based on a surgery schedule, standard facility open hours, occupancy data, and system health data associated with the building equipment, the converged schedule indicating setback period during which the building equipment for an operating room can be operated in a setback state for temperature, airflow, and humidity of the operating room and controlling building equipment based on the converged schedule by causing the building equipment to achieve the compliance requirements for the temperature, pressure, airflow, and humidity of the operating room other than during the setback period during which the temperature, airflow, and humidity are constrained to a setback state during the setback period.

### BRIEF DESCRIPTION OF THE FIGURES

Objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a drawing of a building equipped with a HVAC system, according to some embodiments.

FIG. 2 is a diagram of a system including or interfacing with a BMS, which can be used in the building of FIG. 1, to generate a converged schedule for operation of building equipment, according to some embodiments.

FIG. 3A is a schedule diagram with a converged schedule generated for use with the BMS of FIG. 2, according to some embodiments.

FIG. 3B is a schedule diagram with a converged schedule generated for use with the BMS of FIG. 2, according to some embodiments.

FIG. 4 is a block diagram of a control system, according to some embodiments.

FIG. 5 is a flow diagram of a process for generating a converged schedule for operation of building equipment for one or more areas of a building, according to some embodiments.

FIG. 6 is a flow diagram of a process for operating building equipment to control one or more climate conditions within an interior space of a building based on a converged schedule, according to some embodiments.

FIG. 7 is a flow diagram of a process for operating building equipment to control one or more climate conditions within an interior space of a building based on a converged schedule, according to some embodiments.

FIG. 8 is a flow diagram of a process for operating building equipment to control one or more climate conditions within an interior space of a building based on performance of a control system, according to some embodiments.

### DETAILED DESCRIPTION

Referring generally to the FIGURES, systems and methods for control and analysis for HVAC systems for a healthcare facility or other space are shown, according to some embodiments. More specifically, the system and methods described herein can be implemented to monitor and control parameters of areas within a building or other facility (e.g., a hospital), in order to avoid and/or identify potential compliance issues. As described herein, compliance issues may generally refer to any indication of non-compliance, where one or more parameters of an area or a building do not meet a set of compliance standards (e.g., standard or predetermined values). The parameters generally include one or more of temperature, pressure, humidity, and airflow of a room, area, or building.

For example, certain an operating room of a hospital or other healthcare facility may be expected to be (e.g., based on industry standards) in compliance with certain target ranges or values for building conditions such as temperature, pressure, and/or humidity in order for the operating room to be usable to provide patient care (e.g., for performance of a surgery). However, it can be energy-intensive to maintain the operating room in compliance at all times via continuous operation of an HVAC system serving the operating room. Accordingly, it may be desirable to allow the building equipment for an operating room to operate in a setback state when not in use and/or otherwise turn down HVAC operations or relax settings to reduce energy consumption during out-of-use times for the operating room. However, it can also be inefficient for hospital operations if an operating room is in a setback state at a time when staff intends to perform an operation in the operating room. The teachings herein relate to technology for controlling an HVAC system to substantially ensure that the operating room is provided with compliant environmental conditions when the operating room is to be used, while enabling energy savings during out-of-use time periods.

In some embodiments, the systems and methods described herein may be applied to rooms or spaces within a hospital or another industrial building where temperature, pressure, humidity, and airflow must be monitored and checked for compliance with regulations or process controls. As described above, compliance regulations may include standards set by governmental or non-governmental entities and compliance may be checked by a compliance officer. Checks for compliance of temperature, pressure, humidity, and airflow may be checked randomly or on a set routine or schedule and can affect the ability of the building to continue operation (e.g., an out of compliance hospital room such as an operating room may be inhibited from being used for providing patient care). In some cases, a third party may administer the compliance checks and/or may establish compliance standards.

The systems and methods described herein may continually monitor temperature, pressure, humidity, and airflow measurements from any number of rooms or areas within a building (e.g., a hospital). The environmental conditions data may be used to generate trend data that indicate temperature, pressure, humidity, and airflow measurements for a room or area over time. In some embodiments, the trend data may be analyzed using a predictive model to predict future non-compliance issues. Additionally, in some embodiments, temperature, pressure, humidity, and airflow data from one or more sensors can be compared to a compliance standard to detect compliance issues in real-time. If a room or building falls out of compliance, or if future non-compliance is predicted, automated response process can be implemented. In this regard, the systems and methods described herein for trend analysis and data management can help a facility (e.g., a hospital) maintain compliance standards to decrease downtime due to compliance issues and, in some cases, to decrease or avoid equipment faults. Additional features and advantages of the present disclosure are described in greater detail below.

With continued general reference to the FIGURES, systems and methods are disclosed that improve comfortability for building occupants while maintaining appropriate levels of temperature, pressure, and humidity. In some embodiments, hospitals and/or clinics may need to conform to certain design criteria (e.g., American Society of Heating, Refrigerating and Air-Conditioning Engineers (ASHRAE) standard 170-2017, etc.) with regards to their HVAC systems to minimize infection, maintain staff comfort and contribute to an environment of patient care. These design criteria may require one or more building zones of the hospital or clinic to maintain temperature, pressure, humidity, and airflow within a certain range or ranges. There exists a need to maintain temperature, pressure, humidity, and airflow within these ranges while simultaneously providing comfortability to the building occupants, energy efficiency, and optimization in the HVAC system.

Rooms in hospitals may require special design considerations due to intensified infection concerns (e.g., the spread of a contagious disease, etc.), high air change rates, special equipment, unique procedures, high internal loads and the presence of immunocompromised patients. However, these special considerations may be particularly important for hospital operating rooms (ORs), where their purpose is to minimize infection, maintain staff comfort and contribute to an environment of patient care.

In some embodiments, ANSI/ASHRAE/ASHE Standard 170, Ventilation of Health Care Facilities, is considered a critical standard of heating, ventilation, and air conditioning (HVAC) health-care ventilation design. The intent of the standard may be to provide comprehensive guidance, including a set of minimum requirements that define ventilation system design that helps provide environmental control for comfort, asepsis, and odor in health-care facilities. In some embodiments, it is adopted by code-enforcing agencies.

The standard may define minimum design requirements only, and due to the wide diversity of patient population and variations in their vulnerability and sensitivity, these standards may not guarantee an OR environment that will sufficiently provide comfort and control of airborne contagions and other elements of concern. When selecting the temperature and relative humidity combination to be incorporated into the design, these standard minimums and the desires of the surgical staff may need to be taken into consideration. In some embodiments, the ASHRAE HVAC Design Manual for Hospitals and Clinics discloses the inability to maintain low OR temperature as the primary complaint by surgeons to facility engineers.

### Building With Building Systems

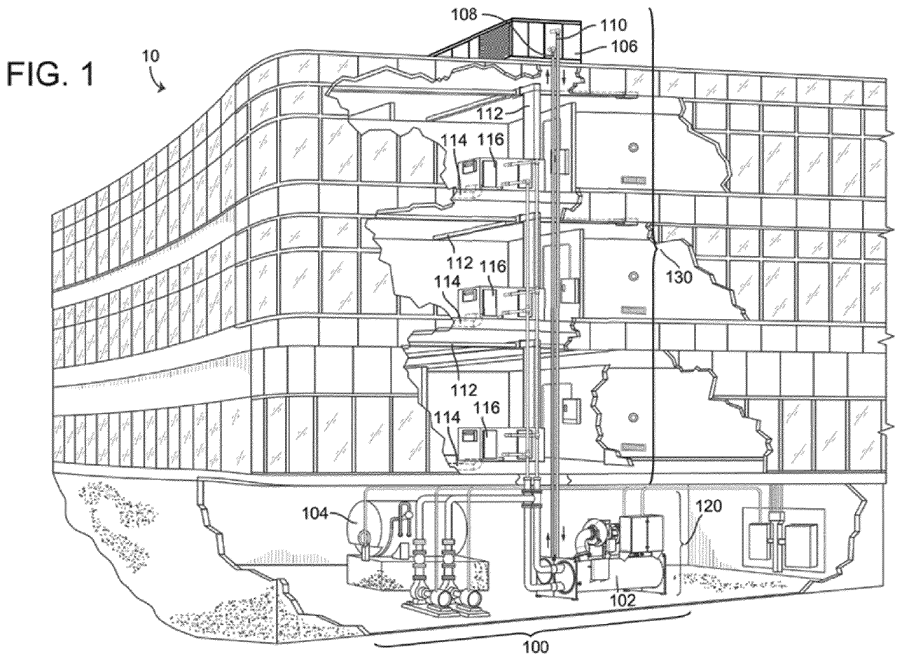

Referring now to FIG. 1, a drawing of a building 100 equipped with a HVAC system 100 is shown, according to some embodiments. More specifically, a perspective view of a building 10 is shown. Building 10 is served by a BMS. A BMS is, in general, a system of devices configured to control, monitor, and manage equipment in or around a building or building area. A BMS can include, for example, a HVAC system, a security system, a lighting system, a fire alerting system, any other system that is capable of managing building functions or devices, or any combination thereof.

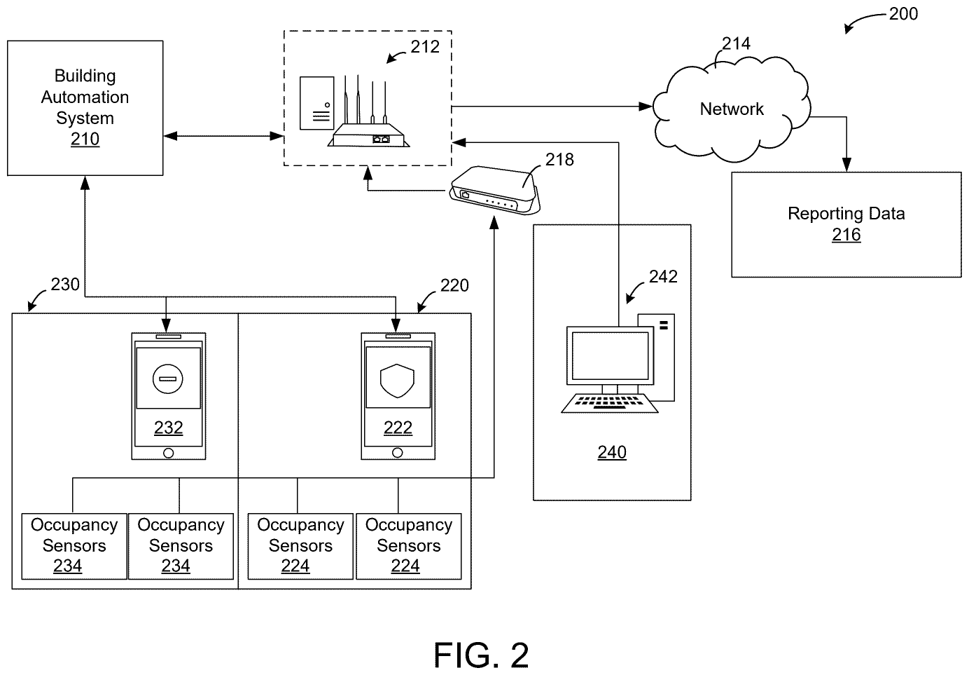

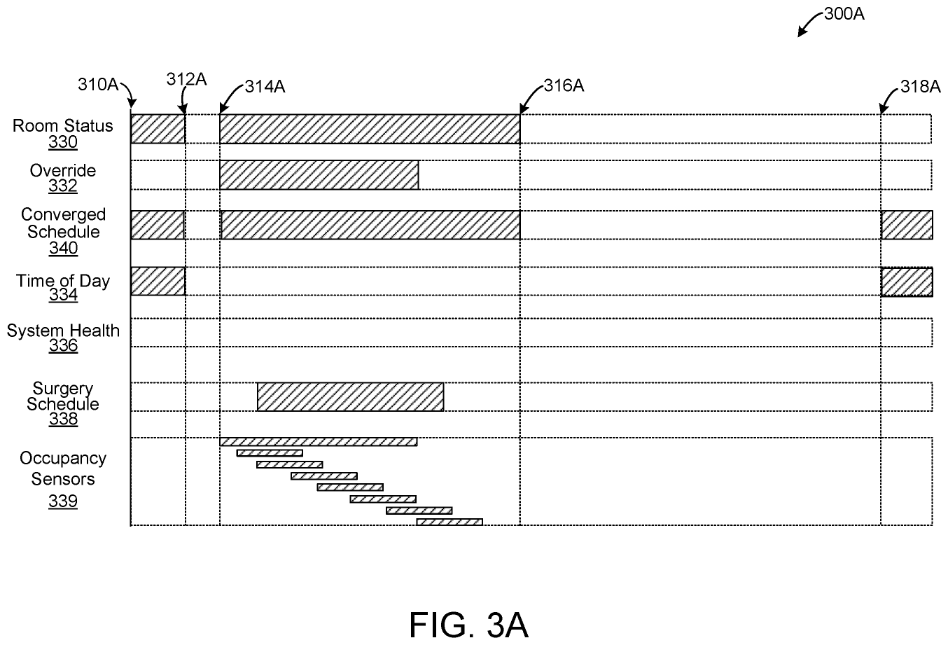

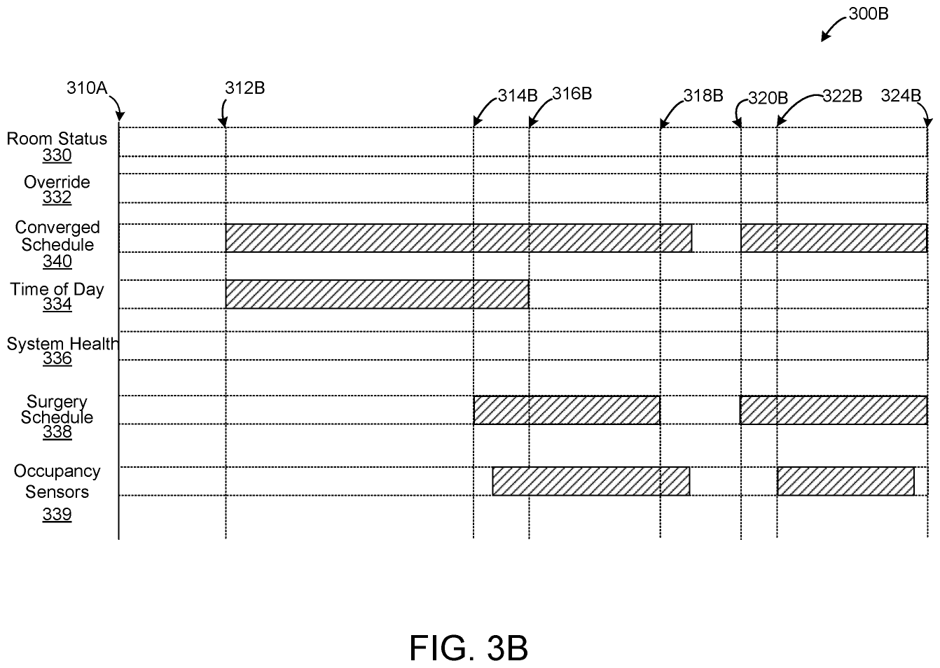

The BMS that serves building 10 includes a HVAC system 100. HVAC system 100 can include a plurality of HVAC devices (e.g., heaters, chillers, air handling units, pumps, fans, thermal energy storage, etc.) configured to provide heating, cooling, ventilation, or other services for building 10. For example, HVAC system 100 is shown to include a waterside system 120 and an airside system 130. Waterside system 120 may provide a heated or chilled fluid to an air handling unit of airside system 130. Airside system 130 may use the heated or chilled fluid to heat or cool an airflow provided to building 10. An exemplary waterside system and airside system which can be used in HVAC system 100 are described in greater detail with reference to FIGS. 2-3.

HVAC system 100 is shown to include a chiller 102, a boiler 104, and a rooftop air handling unit (AHU) 106. Waterside system 120 may use boiler 104 and chiller 102 to heat or cool a working fluid (e.g., water, glycol, etc.) and may circulate the working fluid to AHU 106. In various embodiments, the HVAC devices of waterside system 120 can be located in or around building 10 (as shown in FIG. 1) or at an offsite location such as a central plant (e.g., a chiller plant, a steam plant, a heat plant, etc.). The working fluid can be heated in boiler 104 or cooled in chiller 102, depending on whether heating or cooling is required in building 10. Boiler 104 may add heat to the circulated fluid, for example, by burning a combustible material (e.g., natural gas) or using an electric heating element. Chiller 102 may place the circulated fluid in a heat exchange relationship with another fluid (e.g., a refrigerant) in a heat exchanger (e.g., an evaporator) to absorb heat from the circulated fluid. The working fluid from chiller 102 and/or boiler 104 can be transported to AHU 106 via piping 108.

AHU 106 may place the working fluid in a heat exchange relationship with an airflow passing through AHU 106 (e.g., via one or more stages of cooling coils and/or heating coils). The airflow can be, for example, outside air, return air from within building 10, or a combination of both. AHU 106 may transfer heat between the airflow and the working fluid to provide heating or cooling for the airflow. For example, AHU 106 can include one or more fans or blowers configured to pass the airflow over or through a heat exchanger containing the working fluid. The working fluid may then return to chiller 102 or boiler 104 via piping 110.

Airside system 130 may deliver the airflow supplied by AHU 106 (i.e., the supply airflow) to building 10 via air supply ducts 112 and may provide return air from building 10 to AHU 106 via air return ducts 114. In some embodiments, airside system 130 includes multiple variable air volume (VAV) units 116. For example, airside system 130 is shown to include a separate VAV unit 116 on each floor or zone of building 10. VAV units 116 can include dampers or other flow control elements that can be operated to control an amount of the supply airflow provided to individual zones of building 10. In other embodiments, airside system 130 delivers the supply airflow into one or more zones of building 10 (e.g., via supply ducts 112) without using intermediate VAV units 116 or other flow control elements. AHU 106 can include various sensors (e.g., temperature sensors, pressure sensors, etc.) configured to measure attributes of the supply airflow. AHU 106 may receive input from sensors located within AHU 106 and/or within the building zone and may adjust the flow rate, temperature, or other attributes of the supply airflow through AHU 106 to achieve setpoint conditions for the building zone.

Referring now to FIG. 2, a diagram of a building management system (BMS) 200 is shown, which can be used in the building of FIG. 1 to generate a converged schedule for the operation of building equipment, according to some embodiments.

The BMS 200 shown in FIG. 2 includes a building automation system 210, a bridge 212, a network 214, reporting data interface 216, a local sensor system 218, an operating room scheduling system 240, a first operating room 220 with first occupancy sensors 224 and first override 222, and a second operating room with second occupancy sensors 234 and second override 232.

The building automation system 210 can be configured to automatically control the operation of one or more aspects of an HVAC system of a building (e.g., the HVAC system 100 of the building 10 shown in FIG. 1). For example, the building automation system 210 can control one or more HVAC systems (or portions thereof) that are associated with one or more operating rooms of a hospital. For example, the building 10 may include a first operating room 220 and a second operating room 230. Each operating room 220, 230 can be an interior space that is used to perform one or more surgical procedures. For example, the operating rooms 220, 230 may include one or more different rooms or spaces for which one or more climate conditions are to be maintained during a surgical procedure (e.g., according to one or more hospital requirements for any operating room in which a surgery occurs). For example, a hospital may require a specified temperature, pressure, humidity, and airflow for an operating room while surgery is performed therein. Accordingly, the bridge and/or the building automation system 210 can be configured to control the HVAC system 100 to ensure the climate of the first and second operating rooms remains within the required parameters while the rooms are in use while also minimizing (or reducing) the amount of time the HVAC system 100 is needlessly active in either of the first operating room 220 or the second operating room 230.

In some embodiments, the building automation system 210 can control the HVAC system based on one or more inputs from, or the operation of, one or more other components of the system 200. For example, the building automation system 210 can be coupled with the bridge 212, the first override 222, and the second override 232. More specifically, the building automation system 210 can be communicably coupled with the bridge 212 (e.g., via one or more wired and/or wireless connections) to receive one or more instructions from the bridge 212 to operate the HVAC system of the first and second operating rooms 220, 230 according to the same.

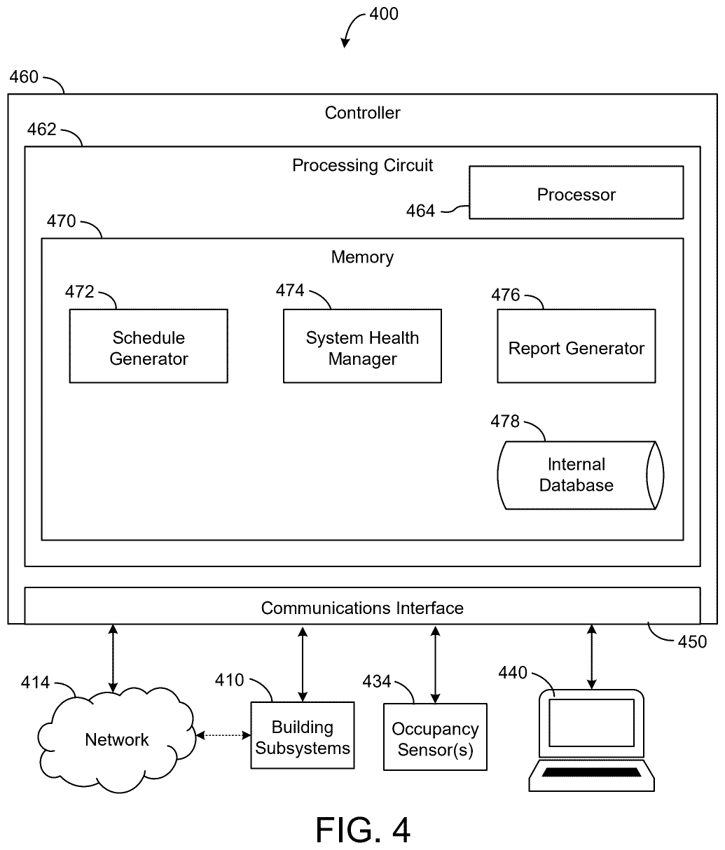

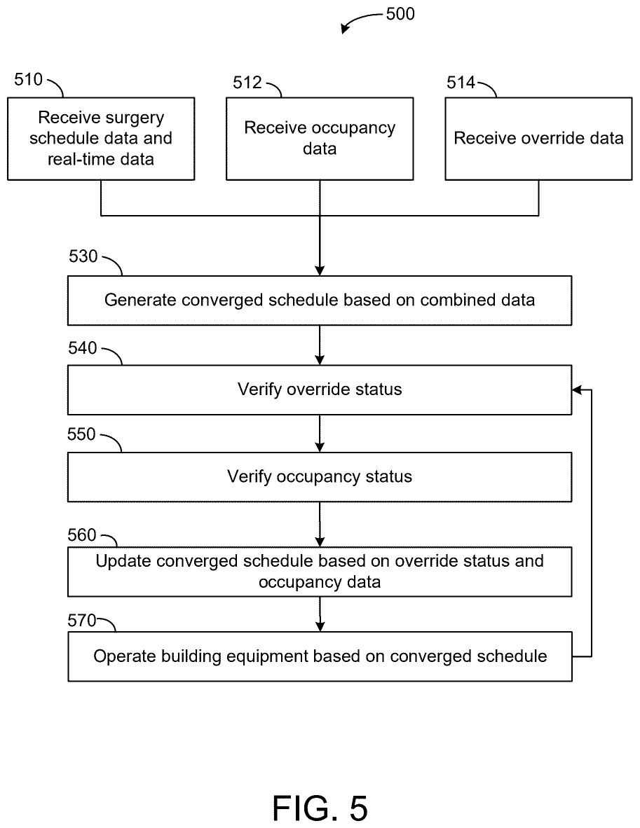

The bridge 212 of the BMS 200 can include one or more processing circuits with one or more memory devices coupled to one or more processing circuits (e.g., as shown in, and described with reference to, FIG. 4). For example, the bridge 212 can include one or more servers or other computer configured to perform one or more functions of the BMS 200 (e.g., receive sensor data, control operation of the building automation system 210, communicate information regarding the status of the BMS to a user, etc.). The bridge 212 can include (e.g., store on the memory device(s)) instructions that, when executed by the one or more processors of the bridge 212, cause the bridge 212 to operate with the building automation system 210 to generate a converged schedule (e.g., described with reference to FIGS. 3A and 3B) and use it to operate the HVAC system 100 for one or more operating rooms (e.g., the first operating room 220 and/or the second operating room 230). For example, the bridge 212 can be configured to implement one or more portions of the methods described below with reference to FIGS. 5-7.

The operating room scheduling system 240 can include surgery schedule data or scheduling information for one or more surgeries planned to occur in either the first or the second operating rooms 220, 230, which can include the dates and times of one or more surgeries scheduled to occur in either of the first operating room 220 or the second operating room 230. For example, when a surgery is scheduled it may be assigned to an operating room (e.g., the first operating room 220) and a date and time may be assigned for that surgery to occur in the assigned operating room. Accordingly, the operating room scheduling system 240 can update the surgery schedule data for that same operating room to reflect the date and time on which the surgery has been scheduled (e.g., update a surgery schedule data of the first operating room 220 to reflect a surgery scheduled for 4:00 PM two weeks from now).

The operating room scheduling system 240 can include one or more local computing devices, which may be housed within the building of the first and second operating rooms 220, 230. For example, the operating room scheduling system 240 can include one or more scheduling servers 242 that are located within (or on the premises of) a hospital that includes the operating rooms that correspond to the scheduling system 240. For example, the surgery scheduling system 240 may be a local scheduling database of the building 10 depicted in FIG. 1.

Alternatively, in some embodiments, the surgery scheduling system 240 can include one or more remote computing devices and/or one or more servers that are located off the premises of the operating rooms associated with the surgery scheduling system 240. For example, a hospital may use a cloud-based scheduling system for the surgeries that will occur in its operating rooms. The surgery scheduling system 240 can include such a cloud-based scheduling system or a portion thereof. For example, the surgery scheduling system 240 can include a single remote database that contains the surgery schedule for the first and second operating rooms 220, 230.

The surgery scheduling system 240 can be configured to communicate the surgery schedule data for each of the first operating room 220 and the second operating room 230 to the bridge 212. Stated differently, the bridge 212 can be configured to receive, or access, surgery schedule data for one or more operating rooms (e.g., the first operating room 220 and the second operating room 230), which can include surgery data maintained by the operating room scheduling system 240 or the scheduling servers. For example, the bridge 212 may be configured with a local connection to the surgery scheduling system 240 (e.g., a wireless and/or wired local network connection between the bridge 212 and the surgery scheduling system 240 with both located on the premises of the first and second operating rooms 220, 230). Alternatively, or in addition, the bridge 212 may be communicably coupled with the surgery scheduling system 240 via one or more networks, including, for example, the network 214. Further, in some embodiments, the bridge 212 may receive surgery schedule data for one or more operating rooms via the internet.

The first occupancy sensors 224 can include one or more sensors configured to detect the presence of an individual within the first operating room 220. The first occupancy sensors 224 can be configured to transmit occupancy data, which reflects the presence or absence of individuals within the first operating room 220, to the bridge 212. In some embodiments, the first occupancy sensors 224 can be configured to transmit the occupancy data to one or more relays or other intermediary that may be coupled between the first occupancy sensors 224 and the bridge 212. For example, the first occupancy sensors 224 may be coupled to a local sensor system 218 that is configured to communicate the occupancy data received from the sensors 224 to the bridge 212. More specifically, the local sensor system 218 can be configured to securely transmit the occupancy data from a local system (e.g., a hospital IT system) to the bridge 212, which may be located remotely (e.g., outside of the hospital of the first and second operating rooms 220, 230).

The first occupancy sensors 224 can be configured with one or more redundant sensors 224, which are configured to ensure complete coverage of the first operating room 220. For example, the first occupancy sensors 224 can include at least two of each sensor needed to detect a person located anywhere within the first operating room. Accordingly, if one of the first occupancy sensors 224 is no longer operational, one or more redundant sensors of the first sensors 224 can still detect whether a person is present in the first operating room 220.

The first occupancy sensors 224 can be configured to collect occupancy data at least once within a specified period of time or at predetermined intervals of time. For example, the first occupancy sensors 224 can collect occupancy data for the first operating room 220 once a minute, once every five minutes, once every ten minutes, and so on up to any amount of time indicated for a collection of new occupancy data. In some embodiments, the first occupancy sensors 224 can be configured to collect occupancy data continuously, or in a substantially continuous manner, such that the occupancy sensors 224 and/or the local sensor system 218 automatically indicate to the bridge 212 whenever there is a change in the occupancy data of the first operating room 220.

For example, the first occupancy sensors 224 can continuously collect occupancy data for the first operating room 220 while it is empty and may send corresponding occupancy data to the bridge 212 (e.g., the local sensor system 218 may indicate to the bridge 212 that the first operating room is empty or in an unoccupied status). Once one or individuals are detected within the first operating room by the first occupancy sensors 224, the first occupancy sensors 224 and/or the local sensor system 218 can automatically send an update, or new occupancy data, to the bridge 212 to indicate the change in the occupancy data (e.g., send occupancy data, such as second occupancy data, to the bridge 212 indicating that the first operating room 220 has changed to an occupied status).

The BMS 200 automatically updates the status of an operating room based on the most recent occupancy data. For example, if the occupancy data indicates a person is present in the first operating room 220 while it is in a standby mode (e.g., if the occupancy data from the first occupancy sensors 224 meets a set of occupancy conditions), the BMS 200 can automatically change the first operating room to an active or ready status (e.g., maintain the climate of the first operating room, via the HVAC system 100, in compliance with hospital standards). Stated differently, the occupancy data, collected by the occupancy sensors 224, 234, can be used for a ‘start trigger’ or to trigger an initial change in an operating room's status (e.g., from a standby, or setback, status to a ready status). The occupancy data can be evaluated over a rolling period of time (e.g., five minutes) and, if the occupancy criteria is met during that time (e.g., occupancy data indicating a person is present for longer than three seconds), the operating room is placed into a ready mode (e.g., the climate kept in compliance with hospital requirements) for a predetermined amount of time (e.g., for three hours beginning at the time the occupancy criteria were first met).

Additionally, in some embodiments, the occupancy data can be used for a maintain trigger or to determine whether to maintain an operating room in a ready state (e.g., continue to maintain the climate in compliance). For example, the BMS 200 can evaluate the occupancy data from the first occupancy sensors 224 over a rolling period of time (e.g., thirty minutes) to determine whether the data meets occupancy criteria. If the occupancy data meets the criteria, the BMS 200 keeps the first operating room 220 in ready mode for a predetermined amount of time (e.g., one hour). Accordingly, in some embodiments, the BMS 200 (e.g., the bridge 212 and/or the building automation system 210) can operate the building equipment (e.g., HVAC system 100) for one or more operating rooms based on an amount of time since the latest occupancy data was collected (e.g., since the time that second occupancy data, indicating a person is present in the first operating room 220, was collected).

Additionally, in some embodiments, the first and second overrides 222, 232 can be directly coupled to the building automation system 210 to operate the HVAC system(s) of the first and second operating rooms 220, 230 without regard to the bridge 212 and its operation. For example, the first and second overrides 222, 232 may be located within the first and second operating rooms, respectively. The overrides may be manually activated by someone located within the respective operating room 220, 230 (e.g., by a user or other individual) and may be configured to ensure the corresponding HVAC system is active and operating to maintain the climate of that operating room within required parameters in response to a user input. For example, the overrides 222, 232 may each operate to control the HVAC system 100 for the first operating room 220 and the second operating room 230, respectively. Each override or override switches 222, 232 can be in communication with the building automation system 210 or, in some embodiments, they may communicate directly with the HVAC system 100. Each override 222, 232 can be disposed proximate to (e.g., inside of and/or near an entrance for) the corresponding operating room 220, 230. The override switches 222, 232 can be thermostat interfaces (e.g., wall thermostats) in the corresponding operating rooms 220, 230, in some embodiments.

As described above, each override 222, 232 may be individually activated by physical interaction with the override or an associated device (e.g., a switch, a touchscreen, keypad, or other input device). For example, the first override 222 may be located within the first operating room 220 and may be activated by a user via physical interaction with a touchscreen of the first override. Once activated, the overrides 222, 232, can cause the building equipment (e.g., the HVAC system 100) to maintain one or more of the temperature, pressure, humidity, or airflow of the operating room(s) within the required parameters for that operating room. In some embodiments, the overrides 222, 232 may cause the HVAC system for the operating rooms 220, 230 to operate until the corresponding override 222, 232 is deactivated (e.g., via user interaction described above for activation of the overrides 222, 232). In some embodiments, the overrides 222, 232 may cause the HVAC system 100 to operate for the corresponding operating room only for a specified duration and may be reactivated to continue to cause the HVAC system 100 to operate for that operating room. For example, upon activation of the first override 222, the HVAC system 100 and/or the building automation system 200 may maintain the first operating room 220 in a ready status (e.g., maintain the climate of the first operating room 220 in compliance with hospital requirements) for approximately three hours.

In one embodiment, the BMS 200 can further include one or more sensors of a climate sensor array, which can be structured to provide sensor data to the BMS 200 (e.g., to the bridge 212). The sensor data collected by the climate sensor array can be indicative of one or more of temperature, pressure, humidity, and airflow in an interior space, including, for example, the first operating room 220 and the second operating room 230. The sensors of the climate sensor array can be used (e.g., by the building automation system 210 and/or the HVAC system 100) to maintain the climate of the operating rooms 220, 230 according to one or more required parameters (e.g., to maintain the temperature, pressure, humidity, airflow etc. of the operating room within a range of required values set by the hospital).

For each operating room of the BMS 200, the bridge 212 can be configured to generate a converged schedule (e.g., as shown in, and described with reference to, FIGS. 3A and 3B). The converged schedule can indicate one or more periods of time during which the corresponding operating room can be switched to an unoccupied turndown or setback state during which the HVAC system can be operated at less-intensive setpoints for one or more of the temperature, pressure, humidity, and airflow of the operating room (e.g., one or more different settings or setpoints than during occupied, in-use, active, etc. periods). For example, the bridge 212 can generate a converged schedule that is updated in real time to reflect relevant (e.g., unexpired) occupancy data and other current inputs relevant to determining whether the operating room requires operation of a corresponding HVAC system. For example, the converged schedule may be updated at a predetermined rate (e.g., every minute) based on the time of day at the operating room's location, the most recent occupancy data in the operating room, the operating room's latest scheduling data, and the status an override signal (e.g., an override device, an override button or override switch that is physically present in, or disposed proximate to, an operating room) associated with the operating room.

The BMS 200 can determine one or more setback times, which are periods of time during which a corresponding operating room can be placed in a standby mode (e.g., the HVAC system 100 can cease to control the climate of that operating room, the HVAC system 100 can move setpoints for the operating room to points which require less energy to maintain such as a higher temperature in the summer or a lower temperature in the winter). The setback times of an operating room can be determined using the converged schedule. For example, the BMS 200 can determine one or more setback times of the first operating room 220 by identifying one or more periods of time in the converged schedule of the first operating room 220 outside of regular operating hours (e.g., between 6:00 PM and 5:00 AM), during which no surgeries are scheduled and the first operating room 220 is unoccupied (e.g., as shown in, and described with reference to, FIGS. 3A and 3B). Alternatively, the setbacks for an operating room (e.g., the first operating room 220) can be determined as part of the converged schedule (e.g., during generation of the converged schedule of the first operating room 220). Accordingly, the BMS 200 can receive new occupancy sensor data indicating occupancy of the first operating room 220 and resume climate control of the first operating room 220 before the end of the setback time in response to the new occupancy sensor data.

In some embodiments, once a person has been detected in an operating room (e.g., via the occupancy data collected by one or more occupancy sensors 224, 234) the bridge 212 can update the converged schedule in that operating room and cause the corresponding HVAC equipment to control the climate within the operating room at least for a specified period of time after the time when the person was first detected in the operating room. For example, a converged schedule may be generated for the first operating room 220. The bridge 212 may update the converged schedule of the first operating room 220 as soon as a person is detected inside of the first operating room 220 and cause (e.g., via the building automation system 210) the HVAC system 100 to maintain the climate within the first operating room 220 within one or more specified parameters at least until thirty minutes after any person has been detected in the first operating room 220.

The bridge 212 can generate each converged schedule based on multiple inputs and other data associated with the operating room for which the converged schedule is generated. For example, the bridge 212 can generate a converged schedule for the first operating room 220 using sensor data of a climate sensor array within the first operating room 220, surgery schedule data (e.g., received from the surgery scheduling system 240), for the first operating room 220, and/or occupancy data received from the one or more first occupancy sensors 224.

As described above, the BMS 200 can operate climate control equipment (e.g., the HVAC system 100) for the first and second operating rooms 220, 230 according to one or more converged schedules generated by the bridge 212, occupancy data collected by the first and second occupancy sensors 224, 234, the first and second overrides 222, 232 (e.g., an override status associated with an operating room), and the time of day corresponding to each of the operating rooms 220, 230. The BMS 200 can include one or more scheduling algorithms, or decision making algorithms, which it may use to generate each converged schedule of an operating room.

In some embodiments, the BMS 200 may determine a minimum amount of time required to achieve one or more required climate conditions within an operating room. For example, the bridge 212 can determine a minimum amount of time before the first operating room 220 can transition from an setback state based on a converged schedule for the first operating room 220, sensor data from one or more climate sensors of the first operating room 220, and one or more climate parameters required for a surgery in the first operating room 220. In some embodiments, the climate parameters for the HVAC system 100 to maintain within one or more operating rooms 220, 230 may be provided to, and/or accessed by, the BMS 200 or one or more portions thereof (e.g., provided as input to the bridge 212, stored in memory of the bridge 212 and/or building automation system 210, received from the surgery scheduling system 240, etc.).

The BMS 200 can generate reporting data that includes the status of the operating rooms 220, 230 and of the building equipment (e.g., the building automation system 210, the HVAC system 100, etc.). For example, the reporting data can indicate a current status of an operating room 220, 230, including whether either operating room is in a standby (e.g., inactive, setback) state or in a ready (e.g., active) state. The reporting data can further include the following for each of the operating rooms 220, 230: the current climate conditions in the room, the current converged schedule, the occupancy status (e.g., whether the room is currently occupied and, if not, the most recent time it was occupied), the duration of one or more periods of time during which the room was, or will be, in a standby mode (e.g., one or more setbacks) and the cost savings associated with the setbacks used for the room over a specified period of time (e.g., the cost savings by automatically placing the room in standby over the past week, the past thirty days, the past three months, etc. as compared to operating the room without such setbacks). The BMS 200 can generate the report based on the sensor data, the occupancy data, and the building equipment and transmit the report to a user device.

Referring now to FIG. 3A, a diagram of a first schedule 300A generated for use with the BMS 200 of FIG. 2, is shown, according to some embodiments. The first schedule 300A includes different types of inputs used to generate the first converged schedule 340, as described above. For example, the first schedule 300A includes room status 330, override or override status 332, the converged schedule 340 (e.g., generated by the BMS 200), time of day 334 for the corresponding operating room (e.g., the first operating room 220) (e.g., standard open hours, indicating that the operating should be available for standard business hours or other preset time-of-day based schedule for facility operations), system health 336 (e.g., the availability of one or more sources of input data for the converged schedule 340, as described in greater detail below), surgery schedule 338 for the corresponding operating room (e.g., the first operating room 220), and occupancy sensor data 339. The first schedule 300A further includes a first time 310A, a second time 312A, a third time 314A, a fourth time 316A, and a fifth time 318A.

More specifically, the first schedule 300A reflects a period of time with a surgery scheduled in the first operating room 220 for 8:00 PM to 12:00 AM local time (e.g., between third time 314A and the fourth time 316A). One or more nurses or other personnel enter the first operating room 220 at 7:00 PM, to prepare for the 8:00 PM surgery, and presses the override switch of the first operating room 220, as reflected in the override 332. Additionally, the occupancy sensor data 339 indicates that the first operating room 220 is occupied beginning at 7:00 PM (e.g., when the one or more nurses or other personnel enter the first operating room 220). Additionally, one or more nurses or other personnel may remain in the first operating room 220 after the surgery has completed, which may be reflected in the occupancy sensor data 339 and show the first operating room 220 is no longer occupied at approximately 1:30 AM. The converged schedule 340 may be generated to keep the first operating room 220 in an active status for a period of time after no more persons are detected (e.g., after the occupancy sensor data 339 no longer indicates one or more persons are present in the first operating room 220). For example, the converged schedule 340 keeps the first operating room 220 in an active status for approximately one hour, from 1:30 AM until the fourth time 316A or 2:30 AM.

The converged schedule 340 can further be generated to reflect the first operating room being placed in an setback status from the fourth time 316A (e.g., after the first operating room 220 is no longer occupied and no surgeries are scheduled in the surgery schedule 338) until the fifth time 318A, which may be when the hours of normal operation begin for the first operating room 220 (e.g., from 6:00 AM to 6:00 PM as described above with reference to FIG. 2). Accordingly, the first schedule 300A shows how the BMS 200 can operate to reduce the amount of time that an operating room (e.g., the first operating room) needlessly spends in an active state. Stated differently, the first schedule 300A reflects a converged schedule 340 that allows the operating room to be placed in a setback, inactive, or standby, state between the fourth time 316A and the fifth time 318A.

For example, the first schedule 300A can show the first time 310A as 5:00 PM local time of the first operating room 220 and the second time 312A as 6:00 PM. The first schedule 300A shows the room status 330, and time of day 334 (e.g., standard open hours of a facility at which times the facility is ordinarily open or otherwise in normal use such as regular business hours), as both being active between the first time 310A and the second time 312A. Accordingly, the converged schedule 340 likewise reflects the first operating room as active between the first time 310A and the second time 312A. The room status 330 and the time of day 334 can both show the first operating room as active between the first and second times 310A, 312A because of a hospital policy to maintain all operating rooms in an active state, or a ready status, between the time of 6:00 AM and 6:00 PM local time.

Similarly, the third time 314A can indicate 7:00 PM local time and the fourth time 316A can indicate 2:30 AM local time. As can be seen in FIG. 3A, between the third time 314A and the fourth time 316A, the first schedule 300A shows the override status 332 as active for a portion of time, the surgery schedule 338 active for another portion of time, overlapping with the time during which the override status 332 is active, and the occupancy sensor data 339 active for the entire period of time between the third time 314A and the fourth time 316A. Accordingly, although the first operating room 220 may not be scheduled for surgery (e.g., the portion of the surgery schedule that is not active between the third time 314A and the fourth time 316A), the converged schedule may nevertheless show the first operating room 220 as active at least because of the occupancy sensor data 339 between the two times 314A, 316A.

Referring now to FIG. 3B, a diagram of a second converged schedule generated for use with the BMS 200 of FIG. 2, is shown, according to some embodiments. Similar to FIG. 3A, the second schedule 300B includes a plurality of different inputs used to generate the second converged schedule 340, according to the inputs described above with reference to FIG. 2. For example, the second schedule 300B includes room status 330, override or override status 332, the converged schedule 340 (e.g., generated by the BMS 200), time of day 334 for the corresponding operating room (e.g., the second operating room 230), system health 336 (e.g., the availability of one or more sources of input data for the converged schedule 340, as described in greater detail below), surgery schedule 338 for the corresponding operating room (e.g., the first operating room 220), and occupancy sensor data 339. The second schedule 300B further includes a first time 310B, a second time 312B, a third time 314B, a fourth time 316B, a fifth time 318B, a sixth time 320B, a seventh time 322B, and an eighth time 324B.

More specifically, the second schedule 300B can reflect a period of time with a first surgery scheduled in the second operating room 230 from 5:00 PM to 8:30 PM local time, or between third time 314B and the fifth time 318B, and further reflect a second surgery scheduled in the second operating room 230 from 10:00 PM to 1:30 AM or from the sixth time 320B to the eighth time 324B. The occupancy sensor data 339 shows that one or more nurses or other personnel enter the second operating room 230 for the first time sometime after the 5:00 PM start time (e.g., at 5:20 PM) of the first surgery (e.g., to prepare for and/or begin the first surgery). The occupancy sensor data 339 further indicates that the second operating room 230 is kept in an occupied state until sometime after the fifth time 318 (e.g., 9:00 PM), which can be based on the design and/or configuration of the BMS 200 (e.g., the BMS may be configured to maintain an operating room in an active state for a predetermined amount of time after anyone is detected in the operating room by the occupancy sensors). As can be seen, once the occupancy sensor data 339 no longer indicates that the second operating room is occupied the converged schedule 340 indicates that the operating room 230 can be placed into a standby state until the sixth time 320B when the second surgery is scheduled to begin.

The converged schedule 340 can further be generated to reflect the second operating room being placed in an setback state, or a standby mode, during which the temperature, humidity, and airflow of the second operating room does not need to be kept in compliance, between the first time 310B (e.g., when the second operating room 230 is no longer occupied and no surgeries are scheduled in the surgery schedule 338) and the second time 312B and between the first and second surgeries (e.g., approximately thirty minutes after the fifth time 318B until the sixth time 320B). Accordingly, the second schedule 300B shows how the BMS 200 can operate to reduce the amount of time that an operating room (e.g., in for the second schedule 300B, the second operating room 230) needlessly spends in an active state or the duration of unneeded temperature, humidity, and airflow compliance for one or more operating rooms. Stated differently, the second schedule 300B reflects a converged schedule 340 that allows the second operating room 230 to be placed in an setback state, or a standby mode for one or more periods of time based on current and past occupancy data 339, the surgery schedule 338, system health 336, the time of day 334, and an override status or override switch 332.

Referring now to FIG. 4, a block diagram of a data management system for the BMS of FIG. 2 and one or more remote systems is shown, according to some embodiments.

As shown in FIG. 4, a block diagram of a controller (e.g., bridge 212 and/or building automation system 210) for controlling HVAC operation, schedule and sensor data management, and report facilitation is shown, according to some embodiments. In general, the controller 460 can automatically monitor and control the parameters of an operating room (e.g., the first and/or second operating rooms 220, 230) over time, and generate reports that improve a compliance review process and indicate an amount of cost savings associated with the amount of time one or more operating rooms spend in a standby mode (e.g., when temperature, humidity, and airflow of the one or more operating rooms is not controlled for compliance). As described above, for example, parameters such as temperature, pressure, humidity, and airflow of a room or rooms can be monitored to efficiently prevent non-compliance. In a hospital setting, for example, predetermined compliance standards may be set by The Joint Commission (TJC) or by the Centers for Medicare & Medicaid Services (CMS), and the controller 460 may at least partially automate the process of monitoring the environmental conditions for rooms or areas of the hospital based on the predetermined compliance standards and compiling compliance reports. Additionally, the controller 460 may determine that one or more parameters (e.g., temperature, pressure, humidity, and airflow) of a room are falling out of compliance, or may become non-compliant in the near future, and can take appropriate actions to avoid non-compliance.

The controller 460 is communicably coupled to the occupancy sensors 434 and the building subsystems 410. In this regard, the controller 460 may receive data regarding one or more parameters of the operating rooms 220, 230 from the occupancy sensors 434, analyze or process the data, and control one or more of the building subsystems 410 based on the data. In some embodiments, the controller 460 may also receive operating data from any of the building subsystems 410, a supplementary system, or a complementary system. In embodiments, the controller 460 may be coupled to the occupancy sensors 434, the building subsystems 410, and the surgery schedule system 440 either directly (e.g., through a wired connection) or indirectly (e.g., via the network 446).

The controller 460 may exchange data with any of the surgery scheduling system 440, the occupancy sensors 434, and the building subsystems 410 via a communications interface 450. The communications interface 450 may be configured to facilitate the exchange (i.e., sending and receiving) of data between the controller 460 and one or more other components. For example, the communications interface 450 may be configured to exchange data via the network 446 and may include appropriate interfaces for communicating on the network 446. For example, the communications interface 450 may include a wired and/or wireless interface for connecting the controller 460 to the Internet, or to an intranet. In some embodiments, the communications interface 450 provides an interface between the controller 460 any one or more the building subsystems, or other components of the BMS 400. In this regard, the communications interface 450 can include a BACnet interface in addition to other types of communications interfaces (e.g., Modbus, LonWorks, DeviceNet, XML, etc.).

In some embodiments, the communications interface 450 facilitates communication between the controller 460 and one or more external databases, including, for example, the surgery scheduling system 440. In some such embodiments, data may be exchanged directly with the surgery scheduling system 440, or indirectly through the network 446. The surgery scheduling system 440 can be implemented in a variety of ways. For example, the surgery scheduling system 440 may include one or more memory devices or remote storage devices. The surgery scheduling system 440 may also include workstations, personal computers, servers (e.g., servers), etc., and may include one or more on-premises server computers/databases and/or one or more cloud-based databases. In this sense, the surgery scheduling system 440 may be distributed across a variety of physical hardware devices.

The communications interface 450 can also facilitates communication between the controller 460 and at least one user device. The user device may be any electronic device that allows a user to interact with the controller 460 through a user interface. Examples of user devices include, but are not limited to, mobile phones, electronic tablets, laptops, desktop computers, workstations, and other types of electronic devices. The user device may be similar to a client device and/or the client devices, as described herein. The user device may display graphical user interfaces or other data on a display, thereby enabling a user to easily view data and interact with the controller 460.

Still referring to FIG. 4, the controller 460 includes a processing circuit 462, which further includes a processor 464 and memory 470. It will be appreciated that these components can be implemented using a variety of different types and quantities of processors and memory. The processing circuit 462 can be communicably connected to the communications interface 450 such that processing circuit 462 and the components thereof can send and receive data via the communications interface 450. The processor 464 can be implemented as a general purpose processor, an application specific integrated circuit (ASIC), one or more field programmable gate arrays (FPGAs), a group of processing components, or other suitable electronic processing components.

The memory 470 (e.g., memory, memory unit, storage device, etc.) can include one or more devices (e.g., RAM, ROM, Flash memory, hard disk storage, etc.) for storing data and/or computer code for completing or facilitating the processes, layers and modules described in the present application. The memory 470 can be or include volatile memory or non-volatile memory. The memory 470 can include database components, object code components, script components, or any other type of information structure for supporting the activities and information structures described in the present application. According to an example embodiment, the memory 470 is communicably connected to the processor 464 via the processing circuit 462 and includes computer code for executing (e.g., by the processing circuit 462 and/or the processor 464) one or more processes described herein.

In some embodiments, the controller 460 is implemented within a single computer (e.g., one server, one housing, etc.). In other embodiments the controller 460 can be distributed across multiple servers or computers (e.g., that can exist in distributed locations). In some embodiments, the controller 460 is embodied in the BMS 400 as described above, and accordingly, the processing circuit 462, the processor 464, and/or the memory 470 may one or more of various processing and/or memory components. Additionally, in such embodiments, the components of the memory 470, described below, may be embodied in the BMS 400. In other embodiments, the controller 460 is a stand-alone device or component not embodied in the BMS 400, and therefore includes its own dedicated processing circuit 462, processor 464, and/or memory 470. In yet other embodiments, the controller 460 is embodied as a portion of the BMS 400, a differently arranged BMS, or a building automation system (BAS), and accordingly may share a processing circuit, processor, and/or memory with any of these other BMSs or BASs.

In some embodiments, receiving data (e.g., temperature, pressure, humidity, and airflow data) from multiple sources or systems (e.g., a network of hospitals) can provide for a more robust data set that can be used to better understand problems and better maintain temperature, pressure, humidity, and airflow compliance. In some embodiments, where the controller 460 predicts future non-compliance issues, as described below, the shared data can improve the accuracy of predictive models and therefore improve response of the controller 460 to temperature, pressure, humidity, and airflow changes or issues.

Referring now to FIG. 5, a flow diagram of a process 500 for generating a converged schedule to operate building equipment of one or more interior spaces of a building is shown, according to some embodiments. The process 500 can be executed by the controller 460, in some embodiments.

At step 510, surgery schedule data and real-time data is received, for example from the surgery schedule system 440. The surgery schedule data may be received by the controller 460 at step 510. The surgery schedule data can indicate times at which one or more surgeries or other medical interventions are scheduled to be performed in an operating room. In other embodiments, the surgery schedule data can refer to other patient occupancy or therapy, for example patient room assignment information for patient rooms, a therapy schedule for a dialysis room or radiation therapy space, an imaging schedule for a radiology space, etc. The real-time data may be received by the controller 460 from any number of patient devices, healthcare practitioner devices, or other devices or systems configured to provide one or more real-time indications regarding a status of a patient or room in which the patient is in. The real-time data can indicate the real-time status of the patient and/or the patient room, such that the controller 460 has one or more indications regarding the real-time status of the patient and/or the patient room. The controller 460 can use the real-time data to generate the converged schedule.

At step 512, room occupancy data is received from occupancy sensors, for example occupancy sensors 232, occupancy sensors 234, or occupancy sensors 434. The occupancy data may be received by the controller 460 at step 512. The occupancy data can indicate whether a room is occupied, a time at which the room was last detected as occupied, a number of occupants detected in a room, etc., in various embodiments.

At step 514, override data is received, for example from the override switch 332. The override data may be received by the controller 460 at step 514. The override data can indicate that an override was selected (e.g., by a user via override switch 332). In other embodiments, the override data can indicate that no override is occurring.

At step 530, a converged schedule is generated by based on a combination of the surgery schedule data, the real-time data, the occupancy data, and the override data. The converged schedule can also based on a default schedule for a facility and/or various other data. The converged schedule provides an indication as to the times as which an operating room (or other healthcare space) should be in compliance with setpoints or other targets (e.g., constraints) for physical conditions in the space (e.g., temperature, pressure, humidity, airflow) (e.g., “On” times, times the room is to be ready for use) and the times at which the operating room can be in a setback state (e.g., “Off” times, setback times) in which compliance with such setpoints or other targets is not required (e.g., times at which different setpoints, targets, ranges, etc. can be used such as less-resource-intensive setpoints, targets, ranges, etc. for one or more environmental conditions). Generating the converged schedule in step 530 can include scheduling a time as an “On” time when any of data sources indicates that the room is in use, e.g., if any of the following are true: the surgery schedule data indicates a surgery is scheduled, the occupancy data indicates the room is occupied, or the override data indicates that a user has manual requested that the room be available for use (or, in some embodiments, the time of day is in a default range such as standard business hours). FIGS. 3A and 3B provide examples of the generation of converged schedules from combined data. A converged schedule is thereby generated which provides a schedule of on periods and setback periods for the building equipment serving an operating room or other healthcare space.

At step 540, an override status is verified. Step 540 can include checking whether an override device has been engaged to override the status for the space indicated by the converged schedule. For example, when the space is in a setback state according to the converged schedule, step 540 can include checking whether an override has been selected to switch the space into an on state.

At step 550, an occupancy status is verified. For example, when the space is in a setback state according to the converged schedule, step 550 can include checking whether occupancy data indicates that the room is occupied or has been recently occupied (e.g., within a present amount of time). In some embodiments, step 550 can include checking for occupancy (via an occupancy) of a neighboring space or other preparatory space which can be expected to be occupied before the room will be used. In some embodiments, step 550 includes use of a redundant sensor in addition to (different than) an occupancy sensor providing data in step 514.

At step 560, an updated converged schedule is generated based on the verified override status and verified occupancy status. Step 560 can include updating the converged schedule by switching a setback time period to an on period in response to an override found in step 540 or occupancy of the space detected in step 550. Accordingly, step 560 can include dynamically updating the converged schedule as time elapses based on verified override status (from step 540) and/or verified occupancy status (from step 550) as time elapses. For example, the converged schedule may be updated to provide an on period for at least a preset duration (e.g., one hour) after the latest override or detected occupancy.

At step 570, building equipment operates in accordance with the updated converged schedule. For example, the controller 460 may uses the updated converged schedule to control the equipment. Operating the building equipment in accordance with the updated converged schedule can include operating the building equipment using feedback control techniques configured to cause physical conditions of the room (e.g., operating room) to be brought to and/or maintained within or near constraints or setpoints for the room for compliance with first requirements for room usage, in response to the converged schedule indicating that the room should be in an on state. Operating the building equipment in accordance with the updated converged schedule can also operating the equipment to achieve relaxed setpoints or constraints on physical conditions of the room (e.g., second requirements for the room)in response to the converged schedule indicating that the room can be in a setback state, for example by reducing an airflow rate, moving a pressure setpoint toward atmospheric pressure (or the pressure of surrounding space), moving a temperature setpoints up (if cooling is being provided) or down (if heating is being provided), or other such adjustment to reduce equipment utilization in the setback state. In some embodiments, one or more environmental requirements are relaxed in the setback state while one or more additional environmental requirements are consistent between the setback state and the on state (occupied state, in-use state, etc.) (e.g., temperature and airflow may be setback while pressure is maintained, in some embodiments). The building equipment is thereby operated based on the updated converged schedule. As illustrated in FIG. 5, process 500 can be iterated to provide an update-to-date updated converged schedule, for example by iterating through verification of override and occupancy status over time. A reliable converged schedule can thereby be provided and used for control of building equipment for an operating room.

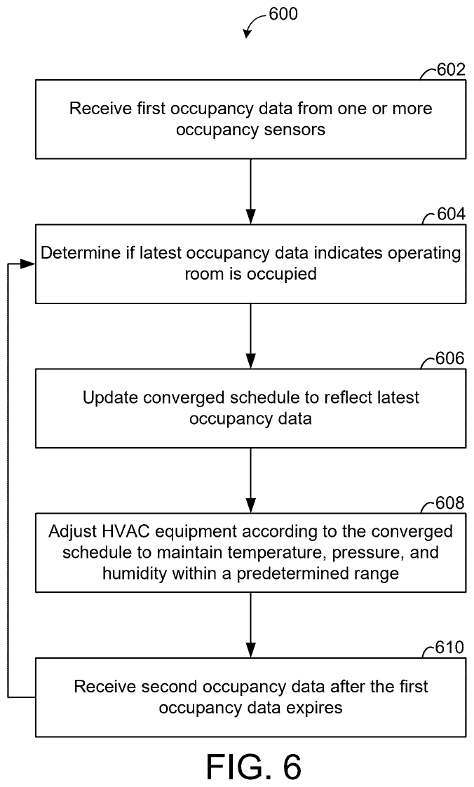

Referring now to FIG. 6, a flow diagram of a process 600 for operating building equipment of one or more interior spaces a building based on a converged schedule is shown, according to some embodiments. The process 600 can be executed by the controller 460, in some embodiments.

At step 602, first occupancy data is received from occupancy sensors, for example, occupancy sensors 224, occupancy sensors 234, or occupancy sensors 434. The occupancy data may be received by the controller 460 at step 602. The occupancy data can indicate whether a room is occupied, a time at which the room was last detected as occupied, a number of occupants detected in a room, etc., in various embodiments.

At step 604, the occupancy data is analyzed to determine the occupancy status of a given operating room, for example to determine whether the latest occupancy data indicates that the operating room is occupied. The occupancy data can indicate whether a room is occupied, a time at which the room was last detected as occupied, a number of occupants detected in a room, etc., in various embodiments.

At step 606, the converged schedule is updated to reflect the latest occupancy data. If the latest occupancy data indicates that the room is occupied but the converged schedule indicates that a current time was expect to be a setback time (e.g., a time when the room would not be in use), the converged schedule can be updated to switch the current time period (e.g., current time step through an upcoming preset duration) to an on state.

At step 608, the updated converged schedule is used to operate HVAC equipment, for example by operating the HVAC equipment to create or maintain the compliant conditions, for example to cause the space to comply with target temperature, pressure, and humidity ranges, in some embodiments. Operating the HVAC equipment in accordance with the updated converged schedule can include providing a first airflow during an active period (e.g., outside of setback periods) and providing a second, lower airflow during a setback period. Other setpoints, setting, power usage, etc. can also be different for the setback period as compared to outside the setback period. In scenarios where occupancy was detected at a time when the converged schedule was indicating a setback state, step 608 can include turning on equipment, updating setpoints to force compliance with required ranges for in use periods (e.g., switching from setpoints required for setback periods), etc., such that the room is brought into compliance for use to perform a surgery or other healthcare operation.

At step 610, second occupancy data is received from occupancy sensors, for example, occupancy sensors 224, occupancy sensors 234, or occupancy sensors 434. The second occupancy data may be received by the controller 460 at step 610. The occupancy data can indicate whether a room is occupied, a time at which the room was last detected as occupied, a number of occupants detected in a room, etc., in various embodiments. Step 610 can include receiving the occupancy data at a point in time at or after the first occupancy data expires, for example such that process 600 is iterating as illustrated in FIG. 6 at regular intervals (e.g., every minute, every fifteen minutes, every hour, etc.). As illustrated in FIG. 6, steps 604-608 can be executed iteratively based on the latest (most recently available) occupancy data.

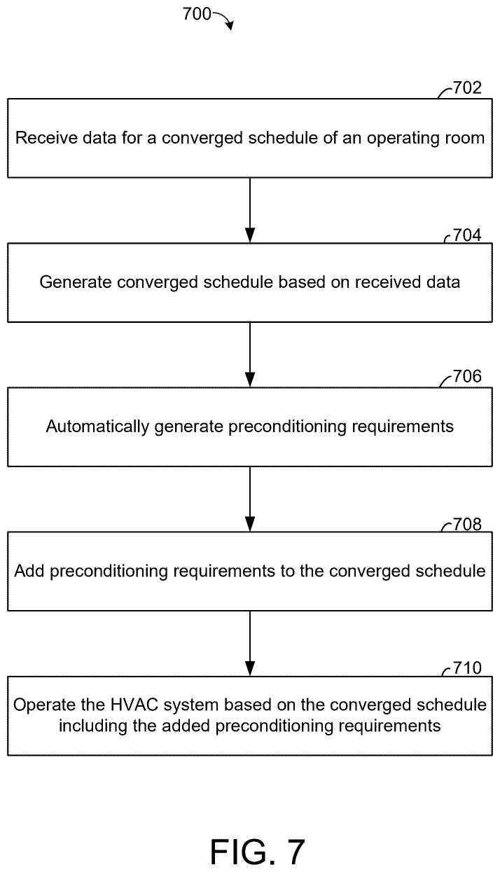

Referring now to FIG. 7, a flow diagram of a process 700 for operating building equipment of one or more spaces of a building (e.g., an operating room or suite of operating rooms) based on a converged schedule is shown, according to some embodiments. The process 700 can be executed by the controller 460, in some embodiments.

At step 702, data for a converged schedule is received. The data for a converged schedule may be received by the controller 460 at step 702. The data for a converged schedule may include the room status data, the override data, the time of day data, the system health data, the surgery schedule data, and the occupancy sensors data, in various embodiments. The data for a converged schedule can indicate when the HVAC system 100 needs to be adjusted and operated.

At step 704, the data for a converged schedule is combined to generate a converged schedule. The converged schedule can be generated according to the various teachings above. The converged schedule may indicate that the room can be placed in a setback status unless any of override data, time of day data, system health data, surgery schedule data, and occupancy data indicate that the room should be in an on state in which conditions are kept ready for surgery, in various embodiments. For example, step 704 can include setting a time period to a setback state if the system health data indicates that one or more sensors or other devices are unavailable, offline, etc. (e.g., if communication is lost to an occupancy sensor).

At step 706, preconditioning requirements are automatically generated. Generating the preconditioning requirements can include determining an amount of time for the physical conditions in a room to be brought back into compliance to transition from the setback state to the on state, for example for temperature, humidity, and airflow to be returned to compliance with temperature, humidity, and airflow ranges to be provided in the space during in-use times (on times) as indicated in a converged schedule. Generating the preconditioning requirements can also include determining settings for the equipment to be used in such preconditioning time periods. Step 706 can be performed using one or more predictive models, for example one or more physics-based models and/or artificial-intelligence models configured to predict amounts of time needed to precondition the space ahead of a time at which the space is to be in compliance according to the converged schedule. In some embodiments, weather forecasts are used as inputs to such models. Such models can be trained (e.g., via machine learning, via a system identification approach for model parametrization, etc.) on historical data associated with the space (e.g., measured temperature, measured pressure, measured humidity, equipment settings, equipment operating data, etc.).

At step 708, the preconditioning requirements are added to the converged schedule. For example, a preconditioning period can be added to the beginning of an “on” period in the converged schedule, wherein the preconditioning period has a length or other characteristic generated as a preconditioning requirement in step 706. The “on” period can considered as having been extended to start earlier in time than it otherwise would have started according to the other converged scheduling features herein, thereby providing for the space to be preconditioned and ready for use at the beginning of the originally-scheduled “on” period. That is, a setback period can be shortened to allow for preconditioning (for moving of conditions from setback conditions to active room conditions).

At step 710, HVAC equipment is operated based on the converged schedule, including based on the added preconditioning requirements. The HVAC equipment be operated to pre-cool, pre-heat, pre-humidify, pre-dehumidify, pre-pressurize, pre-depressurize, etc. the space in accordance with the preconditioning requirements, for example such that the room is in compliance with target conditions at the beginning of a surgery schedule slot, at the beginning of standard business hours, etc., according to various embodiments.

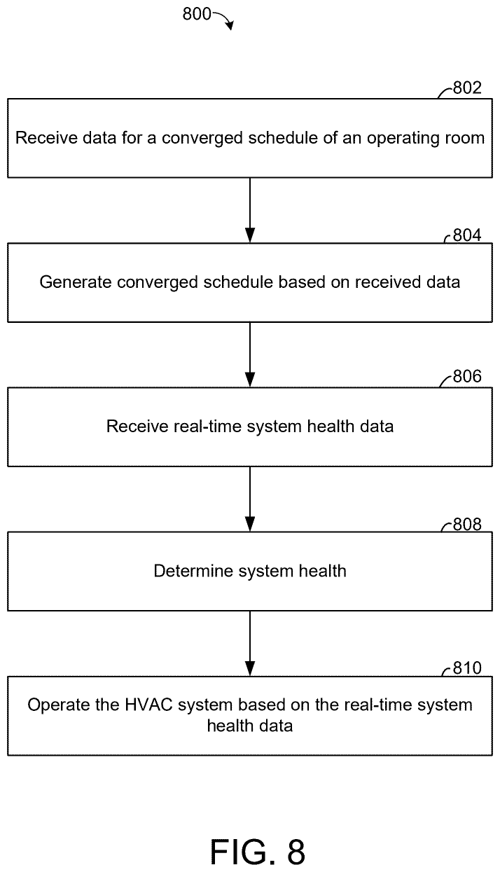

Referring now to FIG. 8, a flow diagram of a process 800 for operating building equipment of one or more interior spaces of a building based on performance of a control system is shown, according to some embodiments. The process 800 can be executed by the BMS 400, in some embodiments.

At step 802, data for a converged schedule is received. The data for a converged schedule may be received by the BMS 400 at step 802. The data for a converged schedule may include the room status data, the override data, the time of day data, the system health data, the surgery schedule data, and the occupancy sensors data, in various embodiments. The data for a converged schedule can indicate when the HVAC system 100 needs to be adjusted and operated.

At step 804, the data for a converged schedule is combined to generate a converged schedule. The converged schedule can be generated according to the various teachings above. The converged schedule may indicate that the room can be placed in a setback status unless any of override data, time of day data, system health data, surgery schedule data, and occupancy data indicate that the room should be in an on state in which conditions are kept ready for surgery, in various embodiments. For example, step 804 can include setting a time period to a setback state if the system health data indicates that one or more sensors or other devices are unavailable, offline, etc. (e.g., if communication is lost to an occupancy sensor).

At step 806, real-time system health data is received. The real-time system health data may be received by the BMS 400 at step 806. The real-time system health data may indicate the performance of the controller 460 and other components configured to generate the converged schedule. The real-time system health data may include information similar to the system health 336 as described herein. The real-time system health data further includes information indicating that the converged schedule is accurate and that the components which generate the converged schedule (e.g., the controller 460, etc.) are operating properly (e.g., generating the converged schedule properly, online and accessible, controlling building equipment properly, etc.).

At step 808, system health is determined. The system health is determined by the BMS 400 based on the real-time system health data. The BMS 400 may compare the real-time system health to the system health 336 of the converged schedule. If the real-time system health and the system health 336 of the converged schedule are consistent (e.g., substantially the same, the real-time value matches the value in the converged schedule, etc.), the BMS 400 may determine that the controller 460 is operating properly, the converged schedule is accurate, and the controller 460 is healthy. If the real-time system health and the system health 336 of the converged schedule are different, the BMS 400 may determine that the controller 460 and the converged schedule are not operating properly, and that the controller 460 is not healthy. In some embodiments, step 808 includes checking whether the converged schedule is being generated and/or provided appropriately and, if some error is detected in generation or provision of the converged schedule, a determination can be made that the system is not healthy.

At step 810, the building equipment is operated based on the real-time system health data. The step 810 may include operating HVAC equipment of the building such that the interior space is in compliance with one or more target conditions. If the system health is determined to be healthy, the controller 460 may operate the building equipment based on the converged schedule, as described above with reference to the step 570. If the system health is determined to not be healthy (e.g., the converged schedule is not operating properly, etc.), the BMS 400 may operate the HVAC equipment to prevent setback. The step 510 may be or include operating the HVAC equipment until the system health is determined to be healthy. For example, the BMS 400 may operate the building equipment to avoid setback until the controller 460 and the converged schedule are operating properly (e.g., the system health is determined to be healthy).

These and other features disclosed in this application enable reliable readiness of an operating room or other healthcare space for use in providing patient care, while also enabling building equipment to be switched to a setback mode when the space will not be in use in order to save energy usage, wear on equipment, etc. as compared to systems which maintain compliance with temperature, pressure, and humidity requirements at all times, among other advantages.

### Configurations of Exemplary Embodiments

The construction and arrangement of the systems and methods as shown in the exemplary embodiments are illustrative only. Although only a few embodiments have been described in detail in this disclosure, many modifications are possible (e.g., variations in sizes, dimensions, structures, shapes and proportions of the elements, values of parameters, mounting arrangements, use of materials, colors, orientations, etc.). For example, the position of elements can be reversed or otherwise varied, and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the connection steps, processing steps, comparison steps and decision steps.

What is claimed is:

1. A system for climate control of an operating room at a healthcare facility, comprising:
building equipment operable to affect at least one of temperature, pressure, airflow, or humidity of the operating room;
a control system programmed to:
generate, based on a converged schedule for the operating room, a setback period during which the building equipment for the operating room can be operated in a setback state; and
control the building equipment using the converged schedule such that the building equipment operates in the setback state during the setback period.

2. The system of claim 1, wherein the control system is programmed to generate the converged schedule by excluding, from the setback period:
first times for which the converged schedule indicates that a surgery is scheduled to be performed in the operating room;
second times corresponding to standard facility open hours; and
third times at which occupancy data indicates the operating room is occupied.

3. The system of claim 1, wherein the control system is programmed to generate the converged schedule by excluding, from the setback period, times at which system health information indicates that a data point relating to the operating room is unavailable.

4. The system of claim 1, wherein the control system is further programmed to:
automatically determine a preconditioning requirement for returning the operating room to a non-setback state following the setback period; and
shorten the setback period in the converged schedule based on the preconditioning requirement.

5. The system of claim 1, further comprising an override device, wherein the control system is further programmed to override the setback period in response to a user input to the override device.

6. The system of claim 1, wherein the control system is further programmed to generate the converged schedule based on system health data associated with the building equipment.

7. The system of claim 1, comprising an occupancy sensor, wherein the control system is programmed to dynamically update the converged schedule based on changes in occupancy data.

8. The system of claim 1, wherein the control system is further programmed to generate a report comprising information on compliance with compliance requirements and energy savings associated with the setback period.

9. The system of claim 1, wherein the control system is programmed to control the building equipment using the converged schedule by reducing an airflow provided by the building equipment during the setback period as compared to at times outside the setback period.

10. The system of claim 1, wherein the control system is programmed to control the building equipment using the converged schedule by:
using a lower temperature setpoint during the setback period as compared to outside the setback period when the building equipment is heating the operating room; and
using a higher temperature setpoint during the setback period as compared to outside the setback period when the building equipment is cooling the operating room.

11. A method for a healthcare space, comprising:
operating building equipment to affect at least one of temperature, pressure, airflow, or humidity of the healthcare space;
generating, based on a treatment schedule for the healthcare space and at least one additional input relating to the healthcare space, a converged schedule for the healthcare space by providing the converged schedule with a setback period during which the building equipment for the healthcare space can be operated in a setback state; and
operating, by the building equipment, in accordance with the converged schedule such that building equipment operates in the setback state during the setback period.

12. The method of claim 11, wherein the at least one additional input comprises occupancy data and standard open hours for the healthcare space, and wherein generating the converged schedule comprises excluding, from the setback period, first times for which the treatment schedule indicates that a treatment is scheduled to be performed in the healthcare space, second times corresponding to the standard open hours, and third times at which a sensed occupancy indicates the healthcare space is occupied.

13. The method of claim 11, wherein generating the converged schedule comprises:
automatically determining a preconditioning requirement for returning the healthcare space to within one or more compliance requirements following the setback period; and
shortening the setback period in the converged schedule based on the preconditioning requirement.

14. The method of claim 11, further comprising:
overriding the converged schedule in response to an override received from a user via an override switch;
dynamically updating the converged schedule based on changes in sensed occupancy from an occupancy sensor; and
automatically generating a report of compliance with compliance requirements and energy savings associated with the setback period.

15. The method of claim 11, wherein operating, by the building equipment, in accordance with the converged schedule comprises providing a lower airflow to the treatment space during the setback period as compared to outside the setback period.

16. The method of claim 11, wherein operating the building equipment to affect the at least one of temperature, pressure, airflow or humidity of the healthcare space comprises heating the healthcare space, and wherein operating, by the building equipment, in accordance with the converged schedule comprises using a lower temperature setpoint during the setback period as compared to outside the setback period.

17. The method of claim 11, wherein operating the building equipment to affect the at least one of temperature, pressure, airflow or humidity of the healthcare space comprises cooling the healthcare space, and wherein operating, by the building equipment, in accordance the converged schedule comprises using a higher temperature setpoint during the setback period as compared to outside the setback period.

18. A system for climate control of an operating room, comprising:
building equipment operable to affect at least one of temperature, pressure, airflow, or humidity of the operating room;
a control system configured to generate a converged schedule for an operating room; and
a building management system (BMS) configured to:
receive real-time system health data;
based on the real-time system health data, determine system health; and
responsive to determining that the system is not healthy, operate the building equipment to prevent the operating room from entering a setback state.

19. The system of claim 18, wherein the controller is further configured to receive real-time data indicating at least one of a real-time status of a patient in the operating room or a real-time status of the operating room.

20. The system of claim 18, wherein operating the building equipment to prevent the operating room from entering the setback state further comprises the BMS operating HVAC equipment of the operating room to prevent the operating room from entering the setback state until the system is determined to be healthy.
